(19) United States
(12) Patent Application Publication
(10) Pub. No.: US 2025/0344965 A1
(43) Pub. Date: Nov. 13, 2025

(54) FORCE PLATFORM SYSTEM USEABLE IN AN ITEM OF FOOTWEAR TO MEASURE THE WEIGHT AND PRESSURE EXERTED BY THE FOOT OF A PATIENT

(71) Applicant: FLUID INNOV, Le Chambon-Feugerolles (FR)

(72) Inventor: Julien EMPEREUR, Tignes (FR)

(73) Assignee: FLUID INNOV, Le Chambon-Feugerolles (FR)

(21) Appl. No.: 18/871,408

(22) PCT Filed: Jun. 21, 2023
(86) PCT No.: PCT/FR2023/050931

§ 371 Date: Dec. 3, 2024

(30) Foreign Application Priority Data
Jun. 22, 2022 (FR) FR22/06142

(51) Int. Cl. A61B 5/103; A61B 5/00; A61B 90/00

(52) U.S. Cl. CPC A61B 5/1038; A61B 5/6807; A61B 5/7271; A61B 5/7405; A61B 5/743; A61B 90/06; A61B 2090/064; A61B 2505/09; A61B 2560/0462; A61B 2562/0247; A61B 2562/04

(57) ABSTRACT

A device for measuring a bearing force exerted by the foot of a patient, the device including: a shell; an inner sole that is intended to support the foot of the patient and that is held in the shell, the shell and the sole being arranged so that, when the foot of the patient is bearing against the inner sole, the forces exerted by the foot of the patient on the inner sole are transmitted to the shell in a limited number of contact regions; sensors that are interposed between the shell and the inner sole in the contact regions, respectively, and that are each configured to deliver a signal representative of a force exerted on the corresponding contact region; and a processing unit connected to the sensors and configured to acquire the force signals delivered by the sensors.

### TECHNICAL FIELD

The present invention generally relates to the field of baropodometry systems used in particular for the rehabilitation of patients who have suffered an injury to a lower limb, or who have a neurological deficiency in the sensitivity of the lower limbs. The present invention relates in particular to a system for measuring the weight applied by a patient to their foot.

### STATE OF THE ART

Patent FR 3 072 016 filed by the Applicant describes a system for measuring the weight exerted by the foot of a patient while walking or standing. This system comprises a footwear item suitable for leg rehabilitation. This system is provided with a sole in which pressure or force sensors are integrated, connected to a processing unit. The processing unit is configured to process the signals from the sensors in real time and provide the patient or rehabilitation therapist in real time information on the weight exerted by the patient foot on the sole.

However, the method for integrating the sensors into the sole and the method for attaching the sole to the footwear does not allow the total weight exerted by the patient's foot on the sole to be measured. Indeed, portion of the patient's weight is transmitted directly from the sole to the footwear and to the ground without being able to be measured by the sensors. The indications likely to be provided in real time by the sensors are therefore erroneous.

It is therefore desirable to propose a footwear article adapted to leg rehabilitation, allowing to measure the total force exerted by a patient's foot on a sole. It is also desirable to be able to indicate in real time to the patient and the rehabilitation therapist information as precise as possible concerning the support forces exerted by the patient's foot and their distribution. It may also be desirable to collect information relating to the distribution of the forces exerted by the patient's foot.

### SUMMARY

Embodiments relate to a device for measuring a support force exerted by a patient's foot, the device comprising: a support, an inner sole intended to support the patient's foot, held on the support, the support and the inner sole being arranged so that, when the patient's foot is bearing on the inner sole, the forces exerted by the patient's foot on the inner sole are transmitted to the support in a finite set of separate contact zones; sensors interposed between the support and the inner sole respectively at the contact zones, and each configured to provide a signal representative of a force exerted on the corresponding contact zone; and a processing unit connected to the sensors and configured to acquire the force signals provided by the sensors.

Thanks to these arrangements, all of the forces exerted by the patient's foot on the inner sole can be measured, which allows better precision to guide the patient in his rehabilitation or neuromuscular stimulation exercises.

According to an embodiment, the device comprises a retaining member arranged between the inner sole and the support in a retaining zone distinct from the contact zones, to secure the inner sole to the support without transmitting the forces exerted on the inner sole to the support.

According to an embodiment, the retaining member is configured to be partially engaged in an orifice through the support and to be fixed in the inner sole, such that the retaining member is retained by the support upwards with a non-zero clearance.

Thus, the sole is retained in the shell without interfering with the force measurements.

According to an embodiment, the support comprises an outer sole of a domed shape along a longitudinal axis of the support.

Indeed, such a domed shape improves training and accelerates the rehabilitation of patients.

According to an embodiment, the sensors comprise three force sensors including a sensor arranged under a rear portion of the inner sole, and two sensors arranged under a front portion of the inner sole.

This arrangement of the sensors makes it possible to precisely locate the center of pressure exerted by the foot on the sole.

According to an embodiment, the support has the shape of a shell with a rim surrounding the sole.

According to an embodiment: the processing unit is configured to calculate in real time a force value applied by the patient's foot on the inner sole from the signals provided by the sensors, and transmit the force value to external equipment, and/or the processing unit is configured to transmit in real time measurements representative of the signals provided by the sensors to the external equipment, the external equipment being configured to calculate in real time a force value applied by the patient's foot on the inner sole from the measurements provided by the processing unit.

The real-time calculation of the exerted force offers the possibility of creating a feedback loop allowing the patient to adjust in real time the force that he exerts on the inner sole.

Embodiments may also relate to a system for rehabilitating a lower limb of a patient, the system comprising: a measuring device as previously defined, and a human-machine interface in communication with the processing unit of the measuring device and configured to emit in real time a signal representative of a force value calculated from the force signals provided by the sensors.

The presence of a human/machine interface providing in real time signals representative of the force exerted on the sole makes it possible to achieve this feedback loop.

According to an embodiment, the human-machine interface comprises a mobile terminal in communication with the processing unit and provided with a dedicated application configured to process signals transmitted by the processing unit.

Thanks to the use of a mobile terminal for example of the “smartphone” type, the creation of a complex human-machine interface can be easily achieved in the form of a dedicated application installed in the terminal.

According to an embodiment, the human-machine interface is configured to emit in real time: a first light and/or sound signal when the force value is comprised in a first range of force values, and a second light and/or sound signal when the force value is comprised in a second range of force values, greater than the first range of force values, and less than a set value.

The production of such simplified stimuli allows the patient to correctly use the system almost instantly.

According to an embodiment, the processing unit or the human-machine interface is configured to calculate in real time the position of a center of pressure located at the barycenter of the forces exerted on the contact zones and measured by the sensors.

The real-time calculation of the position of the center of pressure makes it possible to monitor the evolution of the patient's posture.

According to an embodiment, the human-machine interface is configured to display the position of the center of pressure in relation to the contour of a human foot, and/or variations over time of the position of the center of pressure in the form of chronograms and/or in relation to the contour of a human foot.

The display and real-time updating of the position of the center of pressure allows the patient to make posture corrections, and the therapist to guide the patient in these corrections.

### BRIEF DESCRIPTION OF THE FIGURES

The present invention will be better understood with the aid of the description which follows with reference to the appended figures, in which identical reference signs correspond to structurally and/or functionally identical or similar elements.

FIG. 1 schematically represents, in three-quarter rear perspective, a device for measuring forces exerted by the foot of a patient, according to an embodiment,

FIG. 2 schematically represents, in three-quarter front perspective, the measuring device without an inner sole, according to an embodiment,

FIG. 3 is a schematic exploded perspective view of the device, according to an embodiment,

FIG. 4 schematically represents the device, in section along a vertical median longitudinal plane, according to an embodiment,

FIG. 5 schematically represents a patient equipped with the measuring device,

FIG. 6 schematically represents circuits of a measuring and training system, according to an embodiment,

FIGS. 7A, 7B, 7C and 7D schematically represent a mobile terminal in communication with the measuring system and executing a dedicated application,

FIGS. 8A, 8B represent timing diagrams of variation of the position of a center of pressure of the foot respectively towards the front or the back of the foot, and to the left or right of the foot, during the step,

FIGS. 9A, 9B represent examples of variation curves of the position of the center of pressure of the foot in static mode, displayed superimposed on an image of the outline of a user's foot.

### DETAILED DESCRIPTION

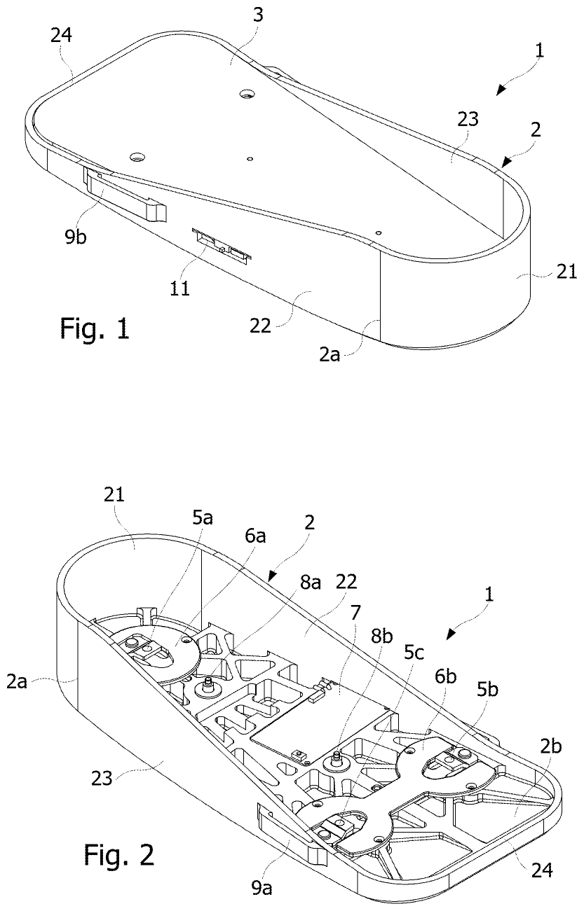

FIGS. 1 to 4 represent a device for measuring forces exerted by a patient's foot, in particular when standing and/or walking. The measuring device is in the form of a shoe 1 comprising a shell 2 supporting an inner sole 3 and including an outer sole 10. The shell 2 comprises a bottom 2b and a rim 2a surrounding the inner sole 3. The rim 2a includes a rear portion 21 (heel side) and lateral portions 22, 23 extending from the rear portion 2a towards the front of the shoe 1, and a front portion 24 (front side of the foot). The height of the rim 2a decreases from the back to the front of the shoe 1 where the rim does not exceed the thickness of the sole 3. The shoe 1 shown in FIGS. 1 to 4 is intended for example for a left foot, or indifferently for a left foot and a right foot. The inner sole 3 is retained in the shell 2 by the rim 2a formed by the portions 21, 22, 23 and 24. The shell 2 can be manufactured by molding a plastic material.

The shell 2 has housings intended to receive sensor supports 6a, 6b. The support 6a is arranged in a housing in the rear region (heel region) of the shoe 1 and supports a sensor 5a. The support 6b is arranged in a front region of the shoe 1 and supports two sensors 5b, 5c, such that the sensor 5b is arranged in a left front region and the sensor 5c in a right front region of the shoe. The inner sole 3 rests exclusively on the sensors 5a, 5b, 5c, such that all of the forces applied to the top of the sole 3 are transmitted to the sensors. Thus, the sensors 5a, 5b, 5c are interposed between the shell 2 and the sole 3 only at the contact zones between the latter, including when the patient exerts all of his weight on the sole 3. The presence of at least three force sensors distributed in the shell 2 makes it possible to prevent the sole 3 from tilting and therefore the sole from bearing on the shell in support zones other than those where the sensors 5a, 5b, 5c are placed.

The shell 2 also has a housing designed to receive an electronic circuit card 7, and a housing designed to receive a battery for powering the card 7. The housing of the battery 7 can for example be accessible via a lateral slot 11 formed in the left portion 22 of the rim 2a of the shell 2. The electronic circuit card 7 is connected to the sensors 5a, 5b, 5c.

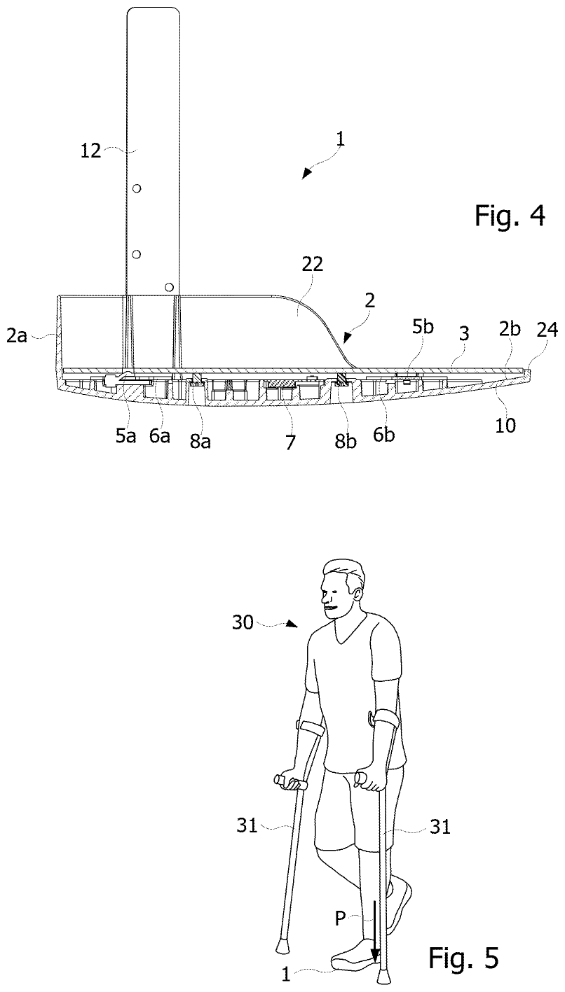

The lateral portions 22, 23 of the rim of the shell 2 can include loops 9a, 9b secured to the outside of the shell, for the passage of straps for holding the shoe 1 on the foot, in the manner of a sandal. In FIG. 4, the shoe 1 can also comprise tabs 12 extending upwards from the lateral portions 22, 22 of the rim 2a of the shell 2 to allow the shoe 1 to be fixed around the patient's ankle.

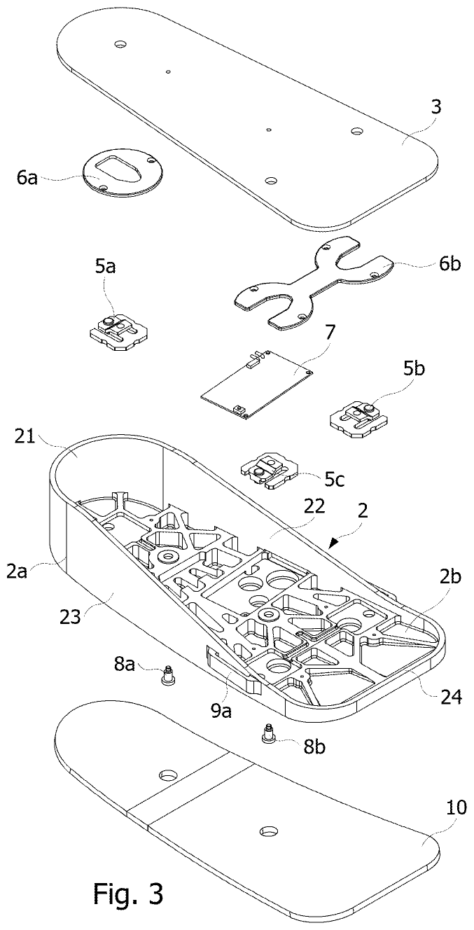

In FIGS. 3 and 4, the inner sole 3 can be held in the shell 2 by at least one screw 8a, 8b. For this purpose, each screw 8a, 8b is inserted into an orifice through the shell 2 to be screwed into the inner sole 3. The head of each screw 8a, 8b is housed in a cavity formed in the shell 2 and opening into the outer sole 10, such that the head of the screw does not bear on the shell 2 or on the outer sole 10 when a pressure is exerted on the inner sole 3. In this way, each screw 8a, 8b only makes it possible to hold the inner sole 3 inside the shell 2 and thus prevent it from falling out of the shell. On the other hand, the screws 8a, 8b do not transmit any force exerted on the inner sole 3 to the shell. In the example of the figures, two screws 8a, 8b are used to hold the inner sole 3, knowing that a single screw may be sufficient and that other screws arranged in this way can be added. It should be noted that it is preferable to leave a certain clearance between the heads of the screws 8a, 8b and the shell 2, in order to prevent any risk of force transmission between the sole and the shell by the screws 8a, 8b. Thus, the sole (3) can be held in the shell with a slight upward clearance.

In FIG. 4, the outer sole 10 is slightly curved along a longitudinal axis of the patient's foot. Indeed, it turns out that such a curved shape improves training and accelerates the rehabilitation of patients. The sensors 5a, 5b, 5c can be of the force sensor type to each measure in real time a force value exerted by a respective area of the patient's foot on the inner sole 3. For example, the force sensors 5a, 5b, 5c are of the strain or pressure gauge type, and are stressed in compression, in a direction perpendicular to the inner sole 3. The strain gauges can be mounted in Wheatstone bridges for greater sensitivity. The strain gauges have many advantages and in particular, they deform little and therefore resist better over time. They also offer high calibration and measurement accuracy, and have a small footprint compatible with that available in the shell, and a reduced cost. Other types of force sensors can be used without departing from the scope of the present description.

FIG. 5 shows a patient 30 equipped with crutches 31, for example due to an injury to one or both lower limbs. The patient 30 has at least one foot 3 equipped with the device 1, to measure the baropodometry of the patient 2.

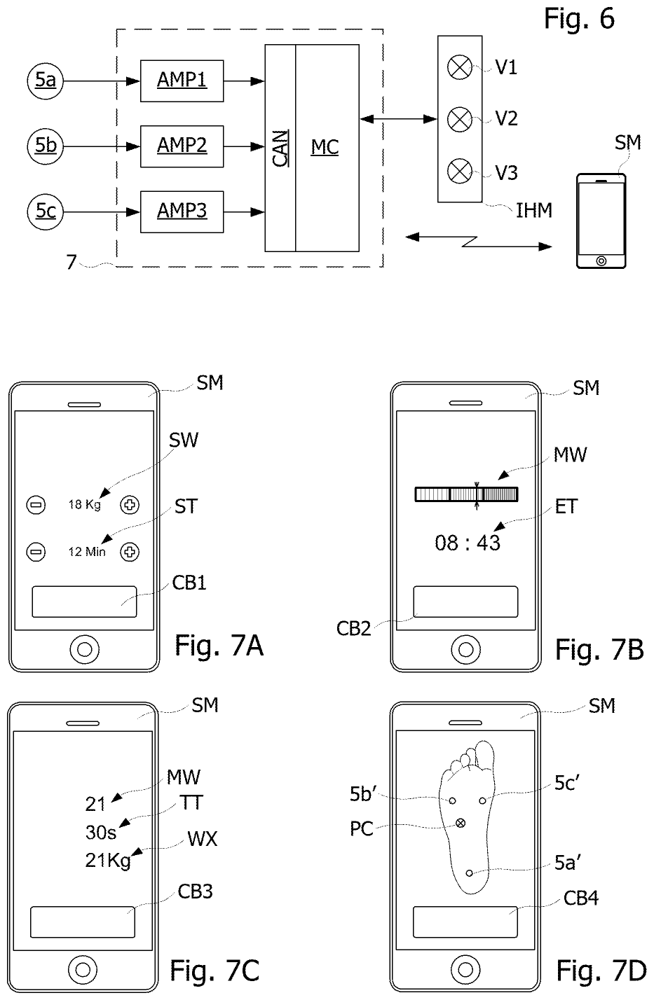

FIG. 6 schematically represents circuits of a lower limb rehabilitation system, according to an embodiment. The system comprises the device 1 provided with the electronic card 7, and therefore the sensors 5a, 5b, 5c integrated in the shell 2, and a human-machine interface HMI. The electronic card 7 supports a processing unit MC and amplifiers AMP1, AMP2, AMP3. Each of the sensors 5a, 5b, 5c is connected to the processing unit MC via conductive connections and a respective amplifier among the amplifiers AMP1, AMP2, AMP3. The measurement signals from the sensors 5a-5c are amplified and transmitted by the amplifiers AMP1, AMP2, AMP3 to analog inputs of the processing unit MC. The processing unit MC converts the received signals using analog/digital converters CAN into digital values which are then processed by the processing unit MC. The processing unit MC can be of the microprocessor or microcontroller type. The electronic card 7 can be powered by a battery or cell which can be inserted into the slot 11. The processing unit MC is connected to the human-machine interface HMI by a wired or preferably wireless communication link, for example of the Bluetooth or BLE (Bluetooth Low Energy) type.

The processing unit MC can be configured to calculate in real time or at a certain rate, a force value P applied by the patient's foot 30 on the inner sole 3 from the measurements provided by the sensors 5a-5c. The calculated force P can be equal to the sum of the forces measured by the sensors 5a-5c. Since all the forces exerted by the patient's foot 30 on the sole 3 are transmitted to the sensors 5a-5c, the value thus calculated by the processing unit MC corresponds to the force P actually exerted by the patient's foot on the sole 3.

Thus, the patient can be constantly informed of the quality of the taken step and thus adapt the next one. For the rehabilitator, real-time access to the information on the taken step allows him to guide the patient and adjust his instructions as the steps progress.

The processing unit MC can also be configured to compare the calculated force P values with a setpoint value. This setpoint value can be adjusted using an adjustment device provided in the interface HMI. Depending on the results of the comparison, the processing unit MC can activate devices V1, V2, V3 for emitting light and/or sound signals, provided in the human-machine interface HMI.

For example, for a certain patient, the set value can be adjusted to 20 kg, and ranges of 18 to 20 kg, 16 to 18 kg, and less than 16 kg can be set. During the patient's walking, throughout the entire step sequence, the device determines in real time the force P exerted on the inner sole 3. When the force P calculated by the processing unit MC is less than 16 kg, the interface HMI does not output a particular signal. When the calculated force P is in the range between 16 and 18 kg, an output device V1 such as a green LED is activated, indicating that the exerted force P is satisfactory. When the force P is in the range between 18 and 20 kg, an output device V2 such as a red LED is activated, indicating that the exerted force P is slightly too high. When the calculated force P is greater than 20 kg, a transmission device such as a sounder V3 is triggered, the transmission device V2 being active, to indicate that the exerted force P is too high and may be harmful to healing.

Thanks to these provisions, the patient 30 can learn to adapt his gait to limit the force exerted by his leg in rehabilitation during walking, in order to promote his healing. The interface HMI thus provides the patient with sensory stimulation allowing neuromuscular feedback stimulating neuroplasticity and sensorimotor reprogramming of the patients.

According to an embodiment, the processing unit MC is configured to calculate in real time the position of a center of support of the foot, corresponding to the barycenter of the forces measured by the sensors 5a, 5b, 5c, and transmit this position to the interface HMI.

According to an embodiment, the processing unit MC or the electronic card 7 is configured to be able to communicate with a mobile terminal such as a «smartphone». FIGS. 7A, 7B, 7C and 7D show such a mobile terminal SM running a dedicated application. The screen of the terminal SM is thus used to emit visual and/or audible signals as part of a human-machine interface.

FIG. 7A shows an image displayed on the screen of the terminal SM having control buttons for setting a target force value SW and an exercise duration ST, and a control button CB1 for validating the setting. Validating the setting using the button CB1 triggers the display of the image shown in FIG. 7B. The image shown in FIG. 7B shows a bar MW indicating different ranges of force values P calculated and transmitted by the processing unit MC and the duration ET of the current exercise. A stop button CB2 displayed on the image of FIG. 7B makes it possible to stop the current exercise. FIG. 7C shows a report of a series of exercises. This report may comprise for example a number of exercises MW, the total elapsed time TT during the exercises and the maximum measured force. FIG. 7D shows an image displayed on the screen of the terminal SM presenting the outline of a human foot, and in relation to the outline of the foot, the respective positions 5a′, 5b′, 5c′ of the sensors 5a, 5b, 5c, and the position of the center of pressure PC calculated by the processing unit MC according to the measurements provided by the sensors 5a, 5b, 5c. The output of this presentation of the position of the center of pressure PC is carried out using a control button CB4.

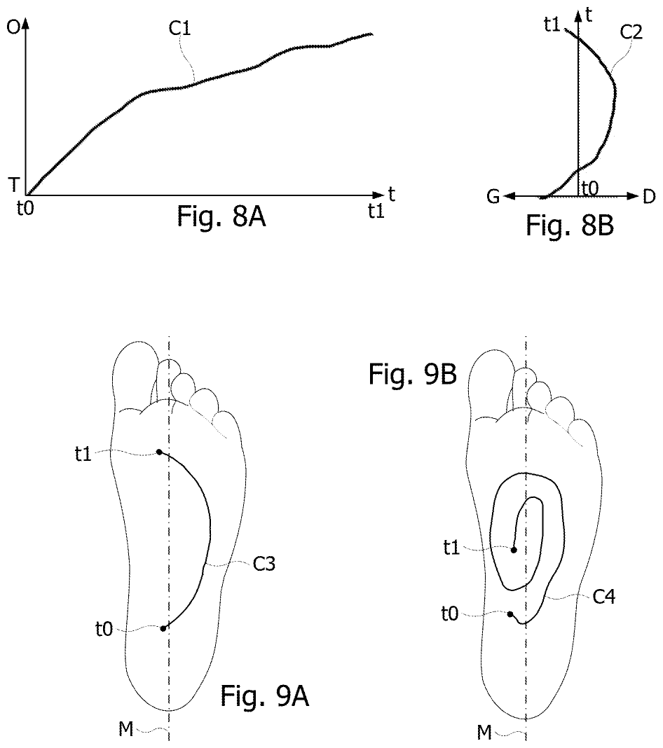

According to an embodiment, the processing unit MC or the electronic card 7 is configured to determine the variations as a function of time of the position of the center of pressure PC of the foot according to the measurements provided by the sensors 5a, 5b, 5c. Furthermore, the processing unit MC can be configured to exploit the variations in the position of the center of pressure PC by displaying curves or timing diagrams. Examples of timing diagrams that can be displayed by the processing unit MC are presented in FIGS. 8A, 8B and 9A, 9B.

Thus, FIGS. 8A, 8B represent timing diagrams of the variation in the position of the center of pressure of the foot during the step, respectively towards O or the back T of the foot, and towards the left G or the right D of the foot. In FIG. 8A, timing diagram C1 shows that the center of pressure PC is gradually displaced from the heel T at time t0 to the toes O at time t1. In FIG. 8B, timing diagram C2 shows that the center of pressure PC is displaced from the left G of a median line of the foot to the right D of this median line, to finally return to the left of the median line.

FIGS. 9A, 9B show examples of curves of variation of the position of the center of pressure of the foot in statics, displayed superimposed on an image of the contour of the foot, between times t0 and t1. In the example of FIG. 9A, the center of pressure is located at time t0 close to the heel and slightly to the left of the midline M of the foot. Then, the center of pressure follows a curve C3 towards an extreme position to the right of the midline M and towards the front of the foot, to end at time t1 in a position close to the toes and slightly to the left of the midline M.

In the example of FIG. 9B, the center of pressure follows a curve C4 in the form of a converging spiral. The curve C4 starts, at time t0, from a position located towards the heel and to the right of the midline M, to go towards a final position at time t1 located towards the center of the foot, slightly to the right of the midline. The curve C4 extends widely to the right and to the left of the foot.

It will be clear to those skilled in the art that the present invention is susceptible to various variants and various applications. In particular, the invention is not limited to a device comprising one or more screws 8a, 8b. Indeed, these screws can be omitted, knowing that they only ensure the maintenance of the inner sole 3 in the shell 2, when the latter is reversed downwards. Furthermore, when the measuring device 1 is used, with the foot of a patient in the shell 2, the inner sole 3 is maintained in the shell by the foot. Moreover, other means may replace the screws 8a, 8b, such as hooks arranged on the shell 2 and cooperating with the edge of the inner sole 3.

It is also not necessary to provide a shell with a rim surrounding the inner sole 3. Only a support for the sensors 5a-5c, the electronic card 7 and the sole 3 may suffice, in particular if screws such as the screws 8a, 8b and their arrangement are provided to secure the sole to the support without transmitting the forces applied to the sole.

Moreover, all or portion of the processing described above as being carried out by the processing unit MC may be carried out by external equipment such as the interface HMI or the terminal SM. Thus, the processing unit MC may simply be configured to calculate in real time a force value applied by the patient's foot from the measurement signals provided by the sensors 5a-5c, and transmit in real time the calculated force value to the external equipment. The processing unit MC can more simply be configured to collect and digitize the measurement signals from the sensors 5a, 5b, 5c, and transmit them to the external equipment. The processing unit MC can even more simply be configured to store the digitized signals from the sensors and, for example in a removable memory card. The memory card can then be read by the external equipment, which can be configured to establish and display an exercise report from the data read from the memory card.

Furthermore, the number of sensors 5a-5c installed in the shell 2 can be other than three, for example greater than three, in particular if a greater precision in determining the position of the center of pressure PC is required.

1. A device for measuring a support force exerted by the foot of a patient, the device comprising:
a support;
an inner sole intended to support the patient's foot, held on the support, the support and the inner sole being arranged so that, when the foot of the patient is bearing on the inner sole, the forces exerted by the foot of the patient on the inner sole are transmitted to the support in a finite set of separate contact zones;
sensors interposed between the support and the inner sole respectively at the contact zones, and each configured to provide a signal representative of a force exerted on the corresponding contact zone; and
a processing unit connected to the sensors and configured to acquire the force signals provided by the sensors.

2. The device according to claim 1, comprising a retaining member arranged between the inner sole and the support in a retaining zone distinct from the contact zones, for securing the inner sole to the support without transmitting the forces exerted on the sole to the support.

3. The device according to claim 2, wherein the retaining member is configured to be partially engaged in an orifice through the support and to be fixed in the inner sole, such that the retaining member is retained by the support upwards with a non-zero clearance.

4. The device according to claim 1, wherein the support comprises an outer sole of a domed shape along a longitudinal axis of the support.

5. The device according to claim 1, wherein the sensors comprise three force sensors including a sensor arranged under a rear portion of the inner sole, and two sensors arranged under a front portion of the inner sole.

6. The device according to claim 1, wherein the support has the shape of a shell with a rim surrounding the sole.

7. The device according to claim 1, wherein:
the processing unit is configured to calculate in real time a force value applied by the foot of the patient on the inner sole from the signals provided by the sensors, and transmit the force value to an external equipment, and/or
the processing unit is configured to transmit in real time measurements representative of the signals provided by the sensors to the external equipment, the external equipment being configured to calculate in real time a force value applied by the foot of the patient on the inner sole from the measurements provided by the processing unit.

8. A system for rehabilitating a lower limb of a patient, the system comprising:
a measuring device according to claim 1, and
a human-machine interface in communication with the processing unit of the measuring device and configured to emit in real time a signal representative of a force value calculated from the force signals provided by the sensors.

9. The system according to claim 8, wherein the human-machine interface comprises a mobile terminal in communication with the processing unit and provided with a dedicated application configured to process signals transmitted by the processing unit.

10. The system according to claim 8, wherein the human-machine interface is configured to emit in real time:
a first light and/or sound signal when the force value is comprised in a first range of force values,
and a second light and/or sound signal when the force value is comprised in a second range of force values, greater than the first range of force values, and less than a set value.

11. The system according to claim 8, wherein the processing unit or the human-machine interface is configured to calculate in real time the position of a pressure center located at the barycenter of the forces exerted on the contact zones and measured by the sensors.

12. The system according to claim 11, wherein the human machine interface is configured to display the position of the center of pressure in relation to the contour of a human foot, and/or variations over time of the position of the center of pressure in the form of chronograms and/or in relation to the outline of a human foot.
